(19) United States
(12) Patent Application Publication
(10) Pub. No.: US 2022/0071196 A1
(43) Pub. Date: Mar. 10, 2022

(54) System and method for cryopreservation of tissues

(71) Applicants: REGENTS OF THE UNIVERSITY OF MINNESOTA, Minneapolis, MN (US); THE UNIVERSITY OF NORTH CAROLINA AT CHARLOTTE, Charlotte, NC (US)

(72) Inventors: Charles Y. Lee, Charlotte, NC (US); John C. Bischof, St. Paul, MN (US); Erik B. Finger, Wayzata, MN (US); Anirudh Sharma, Minneapolis, MN (US)

(21) Appl. No.: 17/421,161

(22) PCT Filed: Jan. 16, 2020
(86) PCT No.: PCT/US2020/013956

§ 371 Date: Jul. 7, 2021

Related U.S. Application Data
(60) Provisional application No. 62/793,535, filed on Jan. 17, 2019.

(51) Int. Cl. A01N 1/02

(52) U.S. Cl. CPC A01N 1/0221; A01N 1/0226

(57) ABSTRACT

A method of preserving tissue includes loading the tissue with a solution with a cryoprotective agent and susceptor particles; freezing the tissue; thawing the tissue; and after freezing and thawing, further processing the tissue. Further processing may include slicing the tissue or digesting the tissue and recovering isolated cells from the digested tissue. The tissue may include at least a portion of an organ. The tissue may include a whole organ. The loading of the solution may be done via the vasculature of the organ. The tissue may be thawed using inductive heating. The susceptor particles may include nanoparticles.

### RELATED APPLICATIONS

This application claims the benefit of U.S. Provisional Application No. 62/793,535, filed on Jan. 17, 2019, the entire teachings of which are incorporated herein by reference.

### FIELD

The present disclosure relates to cryopreservation of tissues. The present disclosure further relates to systems and methods for obtaining large volumes of cells from cryopreserved tissues.

### BACKGROUND

Cell transplantation, bioartificial livers, and engineered livers are promising therapeutic techniques for treatment of end-stage liver disease, acute liver failure, and some liver-based metabolic disorders. At present, these therapies are at different stages of translation to widespread clinical practice. However, although stem cells are a promising solution, there remains a need for an on-demand supply of large numbers of high quality primary human hepatocytes. In addition, the ability to produce any quantity of hepatocytes on demand could also alter testing protocols for drug metabolism and toxicity in the pharmaceutical industry.

Two significant challenges facing obtaining adequate supplies of human hepatocytes are recovery of extended-criteria donor (“ECD”) livers and the need for cryopreservation technologies that yield high quantities (about 10¹¹ cells or greater) of hepatocytes. Currently, ECD livers include elderly, steatotic, and donation after cardiac death (“DCD”) livers and donors with increased risk of disease transmission. Because DCD organs are not harvested until after the cessation of cardiopulmonary function, these organs are commonly associated with injury that results from warm ischemia. Warm ischemia is characterized by a decrease or complete stop of blood flow to at least some organs. The current estimate of the number of cardiac death patients with livers that are not transplantable is greater than 2000 per year. Hepatocytes isolated from DCD livers using conventional technologies have high probability of poor yield, poor viability, and poor functionality. The inability to retrieve viable isolated hepatocytes from available DCD livers limits the availability of these cells for therapies.

Current cryopreservation technologies are not able to produce large quantities of high functioning hepatocytes. Known cryopreservation technologies, which include freezing and vitrification, can preserve about 3×10⁶ cells per 3 mL vial with thawing viability ranging from 50-70%. A single dose of hepatocyte transplantation treatment uses about 2×10⁸ cells per kg of body weight. For a 50 kg patient, this would result in needing to thaw between 4500 to over 6000 vials of cells for one treatment. A bioartificial liver (“BAL”) for treatment requires about 400 g of cell mass to populate the BAL. With approximately 10⁸ hepatocytes per gram of cell mass, the number of vials of cryopreserved cells needed would be prohibitively large and impractical.

Conventional approaches to cryopreservation result in ice crystal formation that damages cells and disrupts the complex macroscopic organization of intact organs. Vitrification involves rapid cooling to a glassy rather than crystalline state, which augments traditional hypothermic preservation and allows biomaterial to be indefinitely stored near the temperature of liquid nitrogen (−140° C.). To achieve the vitrified state, biospecimens must be cooled to the glass transition temperature fast enough to avoid the phase transition of liquid to solid. The cooling rate is termed Critical Cooling Rate (“CCR”) and differs between solutions. On the other hand, the warming rate, termed Critical Warming Rate (“CWR”), is typically at least an order of magnitude higher than the CCR, which means that rewarming from the vitrified state with convective approaches (e.g., a 37° C. water bath) is only possible for small cell and tissue systems (1-3 mL). Addition of cryoprotectants to the system lowers the CCR and CWR, but not enough for larger (>10 mL) tissues and organs.

To preserve cells or tissue samples using vitrification, the specimen is loaded with a vitrification solution. Current vitrification solution loading and unloading protocols occur with either encapsulated cells or cell suspensions. Attachment of primary hepatocytes (˜80%) has been shown to be possible. However, very short exposure time to the cryoprotectants and very rapid cooling and warming rates limit the applicability of the technique to large volumes of cells. A method called liquidus tracking (incrementally lowering the temperature of the aqueous mixture with cells as the vitrification solution is loaded to reduce the toxic effects of the cryoprotectants) has also been applied to encapsulated HepG2 cells. The process of liquidus tracking is described by E. Puschmann et al. in Liquidus Tracking: Large Scale Preservation of Encapsulated 3-D Cell Cultures Using a Vitrification Machine (2017) Cryobiol. 76, 65. Although the technique, as reported, could achieve a viability or about 90%, the process occurred over 11 days, which would not be possible with primary cells, as they would dedifferentiate.

Microwave technology has been studied as a potential method to achieve high warming rates. However, it was found to be unsuccessful due to “hot spots” and subsequent cracking and thermal non-uniformity. Even with the use of uniform fields, inhomogeneous heating occurred due to variations in the dielectric properties of water vs. non-water tissue components (proteins, lipids, DNA etc.), attenuation of the field (skin depth), and even the shape of the sample.

There remains a need for a system and method capable of yielding large amounts of primary cells from cryopreserved tissues on demand.

### SUMMARY

A method of preserving tissue includes loading the tissue with a solution comprising a cryoprotective agent and susceptor particles; freezing the tissue; thawing the tissue; and after freezing and thawing, further processing the tissue. Further processing may include digesting the tissue; and recovering isolated cells from the digested tissue. Further processing may include slicing the tissue. The tissue may include at least a portion of an organ. The tissue may include a whole organ. The tissue may include at least a portion of a liver. The tissue may include a whole liver. The loading of the solution may be done via the vasculature of the tissue. The tissue may be thawed using inductive heating. The susceptor particles may include nanoparticles.

A method of preserving tissue may include perfusing a biospecimen comprising at least a portion of an organ; loading the biospecimen with a solution comprising a cryoprotective agent and susceptor particles; freezing the biospecimen; thawing the biospecimen by inductively heating the susceptor particles; and after freezing and thawing, further processing the biospecimen. Further processing may include digesting the biospecimen; and recovering isolated cells from the digested biospecimen. Further processing may include slicing the biospecimen. The biospecimen may be a whole organ. The organ may be a liver. The loading of the solution may be done via the vasculature of the organ. The susceptor particles may include nanoparticles.

The cryoprotective agent may include one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof. The cryoprotective agent may be present at a concentration of 1M to 10 M. The alcohol may be present at a concentration of 0.5 M to 10 M, or 1 M to 8 M. The sugar may be present at a concentration of 0 M to 3 M, or 0.1 M to 2 M. The cryoprotective agent may include 1 M to 8 M alcohol and 0 M to 2 M sugar. The cryoprotective agent may include 1 M to 8 M ethylene glycol and 0.1 M to 2 M sucrose.

A perfusion solution may include from 0.1 mM to 10 mM of a membrane stabilizer; from 0.1 mM to 15 mM of a metabolic support agent; from 0.1 mM to 10 mM of an antioxidant agent selected from the group consisting of beta-carotene, catalase, superoxide dismutase, dimethyl thiourea (DMTU), diphenyl phenylene diamine (DPPD), mannitol, cyanidanol, a-tocopherol, desferoxamine, 6-hydroxy-2,5,7,8-tetramethyl chroman-2-carboxylic acid, and N-acetyl cysteine, and combinations thereof; a cryoprotective agent; and susceptor particles. The susceptor particles may include nanoparticles. The susceptor particles may include iron oxide. The susceptor particles may include iron oxide nanoparticles. The cryoprotective agent may be present at a concentration of 1M to 10 M and include one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof.

### BRIEF DESCRIPTION OF DRAWINGS

FIGS. 1A-1C show micrographs of hepatocytes of Example 1, plated from control livers (1A) (no loading and unloading of vitrification solution), group 1 (1B), and group 2 (1C).

FIGS. 2A-2C show liver and histology images of Example 2 prior to loading with vitrification solution and susceptor particles (2A), loaded with vitrification solution and susceptor particles (2B), and unloaded vitrification solution and susceptor particles (2C). Blue arrows in FIG. 2B indicate susceptor particles (small blue dots).

FIGS. 3A and 3B are micro-CT images of control (no susceptor particles) and cryoprotective agent—susceptor particle loaded livers in Example 2. Susceptor particles are seen as delineating signal in the blood vessels.

FIG. 4 shows an image of a liver arranged for three-cannula perfusion in Example 3, and a graphical representation of the temperature in the portal vein (PV), suprahepatic vena cava (SHVC) and infrahepatic vena cava (IHVC) during cooling and inductive rewarming.

FIGS. 5A-5C are histology images of the control liver (5A), vitrification solution loaded and unloaded (5B), and vitrified and inductively rewarmed liver (5C) of Example 3. The magnification is 40×.

FIGS. 6A-6C are images of a whole rat liver during normothermic reperfusion with ICG in Example 4 at 0 min (6A), at 30 min (6B), and at 60 min (6C) after vitrification, inductive rewarming, and unloading of vitrification solution.

### DETAILED DESCRIPTION

The present disclosure relates to systems and methods for cryopreservation of tissues. The present disclosure relates to systems and methods for obtaining large volumes of cells from cryopreserved tissues. The systems and methods of the present disclosure may be useful for cryopreserving many tissue types and for obtaining cells from such tissues, such as liver, heart, kidney, pancreas, nerve, and others. In particular, the present disclosure relates to systems and methods for obtaining large volumes of isolated hepatocytes from cryopreserved liver tissue. The systems and methods of the present disclosure may be useful for production of pools of hepatocytes from many donors on demand. In addition, large amounts of high quality plated or suspended hepatocytes can be available to end users on demand.

The systems and methods of the present disclosure may be used to recover and produce large quantities (e.g., 10¹⁰ or greater) of isolated cells, such as hepatocytes. For example, the systems and methods of the present disclosure may be used to recover and produce large quantities of functioning primary human hepatocytes from DCD livers. In preliminary studies, an inductively rewarmed vitrified liver exhibited hepatocyte-specific function and homogeneous flow during normothermic reperfusion. The systems and methods of the present disclosure may also be used to vitrify portions of livers, allowing for greater flexibility in the pharmaceutical industry drug metabolism and toxicity testing programs.

The systems and methods of the present disclosure may be used to combine the technologies of perfusion to resuscitate and condition organs (including DCD livers), infusing the organs with susceptor particles (for example, nanoparticles), vitrifying the organs, and inductively rewarming the organs to avoid recrystallization during rewarming. The ex vivo loading and unloading of the cryopreservation solution using the organ's own native vascular system ensures homogeneous distribution and removal of cryoprotectants and susceptor particles. In addition, the perfusion technology allows resuscitation of DCD livers that have experienced extended periods of warm ischemia, allowing the recovery of hepatocytes that show excellent viability and plating efficiency in animal models. The systems and methods of the present disclosure provide a way to resuscitate DCD livers, vitrify whole and/or partial livers for storage, and when needed, inductively rewarm the livers and isolate any quantity of highly functioning hepatocytes. These large quantities of highly functioning cells would then be available on demand for end users.

The term “organ” is used in this disclosure to describe any organ of a human or an animal. Examples of organs include but are not limited to liver, kidney, heart, pancreas, lung, etc.

The term “portion of an organ” is used here to refer to an intact portion of an organ that includes 1,000,000 cells or more. A portion of an organ, as the term is used here, includes the vascular architecture of the organ, including intact parenchymal cells. The term “intact” in this context means that the cells are connected to each other in the same manner that they would be in the organ. That is, the cells have not been removed, for example, by digestion of the tissue.

The term “nanoparticle” is used here to refer to particles in the size range of 1 nm to 1000 nm.

The terms “normothermic” and “normothermically” are used here to refer to body temperature, about 37° C.

The term “substantially” as used here has the same meaning as “significantly,” and can be understood to modify the term that follows by at least about 75%, at least about 90%, at least about 95%, or at least about 98%. The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

According to an embodiment, a method for preserving tissue includes loading the tissue with a cryopreservation solution that includes a cryoprotective agent and susceptor particles, and freezing the tissue. The terms “cryopreservation solution” and “vitrification solution” are used here interchangeably. The method may further include inductively rewarming (e.g., thawing) the tissue by using an alternating magnetic field to activate the susceptor particles. The tissue may be a whole organ or a portion of an organ. Examples of organs that may be preserved using the method include liver, heart, kidney, pancreas, nerves and nerve tissue, etc. In some embodiments, the organ or portion of an organ has its vasculature substantially intact.

The cryoprotective agent may include one or more of an alcohol, a sugar, or another cryoprotective compound. Examples of suitable alcohols and cryoprotective compounds include glycerol, sorbitol, ethylene glycol, propylene glycol, inositol, xylitol, mannitol, arabitol, ribitol, erythritol, threitol, galactitol, pinitol, and combinations thereof. The alcohol may be present at a concentration of 0.5 M or greater, 1 M or greater, 2 M or greater, 3 M or greater, 4 M or greater, 5 M or greater, or 6 M or greater. The alcohol may be present at a concentration of 10 M or less, 9 M or less, 8 M or less, 7 M or less, 6 M or less, or 5 M or less. Examples of suitable sugars include sucrose, trehalose, maltose, lactose, fructose, glucose, dextran, melezitose, raffinose, nigerotriose, maltotriose, maltotriulose, kestose, cellobiose, chitobiose, lactulose, and combinations thereof. The sugar may be present at a concentration of 0 M or greater, 0.1 or greater, 0.2 or greater, 0.3 or greater, 0.5 M or greater, or 1 M or greater. The sugar may be present at a concentration of 3 M or less, 2.5 M or less, 2 M or less, or 1.5 M or less. Examples of other cryoprotective compounds include dimethyl sulfoxide (DMSO), and cryoprotectant mixtures such as DP6, VS55, M22, and mixtures and modifications thereof. The other cryoprotective compounds may be present at a concentration of 0 M or greater, 0.5 M or greater, or 1 M or greater. The other cryoprotective compounds may be present at a concentration of 6 M or less, 4 M or less, 2 M or less, or 1 M or less. In some embodiments, the cryopreservation solution may include 1 M to 8 M alcohol and 0 M to 2 M sugar. In one embodiment, the cryopreservation solution includes 1 M to 8 M ethylene glycol and 0.1 M to 2 M sucrose.

The cryopreservation solution may be free of DMSO. The cryopreservation solution may be substantially free of DMSO. For example, the cryopreservation solution may include less than a cryopreservative amount of DMSO. The cryopreservation solution may include 1 M to 8 M alcohol and 0 M to 2 M sugar and be substantially free of DMSO.

The loading of the cryoprotective agent may be performed stepwise by increasing the concentration of the cryoprotective agent in the solution during the process. For example, the loading may be done in two or more steps, where the concentration of the cryoprotective agent is increased to its eventual concentration in the cryopreservation solution. Each step may have a duration of 2 min or longer, 5 min or longer, or 10 min or longer. Each step may have a duration of 60 min or less, 45 min or less, 30 min or less, or 20 min or less. In some embodiments, the loading of the cryoprotective agent is done in three steps with increasing cryoprotective agent concentrations, and where in the last step, the susceptor particles are included in the cryopreservation solution.

Loading may be performed at a hypothermic temperature (below normal body temperature) or at a sub-zero temperature. In some embodiments, the loading is performed at a temperature of 20° C. or lower, 10° C. or lower, 4° C. or lower, 2° C. or lower, or 0° C. or lower. In some embodiments, the loading may be performed by decreasing the temperature as the concentration of the cryopreservation solution increases. For example, during a first step, the cryopreservation solution has a first concentration of cryoprotective agents and the loading temperature is held at about 4° C. During a second step, the cryopreservation solution has a second concentration of cryoprotective agents that is greater than the first concentration, and the loading temperature is held at about 0° C. or lower. The loading temperature may be decreased gradually or step-wise during the loading.

According to an embodiment, the susceptor particles include particles capable of heating when subjected to an alternating magnetic field. In other words, the susceptor particles are capable of inductive heating. The susceptor particles may include any material suitable for inductive heating, such as magnetic particles. Such particles may include, for example, iron, nickel, cobalt, or a combination thereof. In some embodiments, the susceptor particles include iron oxide. In one embodiment, the susceptor particles include an iron oxide core coated with mesoporous silica followed by polyethylene glycol and trimethoxysilane. The susceptor particles may have any suitable particle size. For example, the susceptor particles may have a particle size of 1 nm or greater, 5 nm or greater, 10 nm or greater, 20 nm or greater, 40 nm or greater, 75 nm or greater, or 100 nm or greater. The susceptor particles may have a particle size of 100 μm or smaller, 50 μm or smaller, 20 μm or smaller, 10 μm or smaller, 1 μm or smaller, or 0.1 μm or smaller. In one embodiment, the susceptor particles are nanoparticles with a particle size of 10 nm to 0.1 μm. In one embodiment, the susceptor particles are coated iron oxide nanoparticles with a particle size of 10 nm to 0.1 μm.

The amount of susceptor particles loaded into the tissue (e.g., organ) may be determined based on various factors, such as a target warming rate and the nanoparticles' specific absorption rate (SAR) for the available field conditions. The target warming rate may be set at or slightly above the critical warming rate (CWR) of the cryoprotectants used. For example, the CWR for a 40 wt-% ethyl glycol/sucrose cryoprotectant is 45-50° C./min, and the target warming rate may be set at above 50° C./min (e.g., 55-60° C./min). The SAR for a given nanoparticle may be determined through calorimetry, and gives the heating rate per unit mass of the nanoparticle under constant magnetic field and frequency conditions. Based on these parameters, the amount of nanoparticles used in the loading may be calculated.

According to an embodiment, the method includes perfusion of the tissue. Perfusion may include using a perfusion apparatus. The perfusion apparatus may be used to load the tissue with the cryopreservation solution. The use of a perfusion apparatus generally is described in U.S. Pat. No. 8,802,361 to Lee et al. The perfusion apparatus may be arranged to continuously circulate or move (for example, pump) the solution through a chamber, where the tissue is placed in the chamber, to infuse the solution through the arterial and/or venous vascular system of the tissue, and/or to submerge the tissue in the moving solution. The perfusion apparatus may be arranged to maintain a hypothermic condition. For example, the perfusion apparatus may be arranged to maintain the tissue at a temperature below 37° C., or from 0° C. to 20° C., from 0° C. to 15° C., from 0° C. to 10° C., or from 0° C. to 7° C.

The cryopreservation solution may include a preservation solution, such as a University of Wisconsin (UW) solution (see, e.g., U.S. Pat. Nos. 4,798,824 and 4,879,283) or a Euro-Collins solution (see Squifflet J. P. et al., Transplant. Proc. 13:693-696, 1981). The cryopreservation solution may further include one or more metabolic agents, antioxidant agents, and membrane stabilizer agents. An example of a cryopreservation solution prepared with UW solution and metabolic agents, antioxidant agents, and membrane stabilizer is given in TABLE 1 below.

TABLE 1
UW Solution with added metabolic agents, antioxidant agents,
and membrane stabilizers at exemplary Concentration ranges.

| | |
|---|---|
| Lactobionate | 90 mM to 110 mM |
| Potassium | 90 mM to 110 mM |
| Sodium | 20 mM to 30 mM |
| adenosine | 0.5 mM to 10 mM |
| magnesium Sulphate | 4 mM to 15 mM |
| potassium phosphate, e.g., KH2PO | 15 mM to 30 mM |
| raffinose | 25 mM to 35 mM |
| allopurinol | 0.5 mM to 4 mM |
| glutathione | 1 mM to 10 mM |
| metabolic support agent | 0.5 mM to 10 mM |
| membrane stabilizer | 0.5 mM to 10 mM |
| antioxidant agent | 0.1 mM to 10 mM |

Examples of suitable metabolic support agents include glucose, glutamine, lactate, pyruvate, lysine, and combinations thereof. The metabolic support agents may be present at a concentration of 0.1 mM or greater, or 1 mM or greater. The metabolic support agents may be present at a concentration of 10 mM or less, or 5.5 mM or less.

Examples of suitable membrane stabilizers include calcium, glycine, chlorpromazine, and combinations thereof. The membrane stabilizer may be present at a concentration of 0.1 mM or greater, or 1 mM or greater. The membrane stabilizers may be present at a concentration of 10 mM or less, or 5.5 mM or less.

Examples of suitable antioxidant agents include beta-carotene, catalase, superoxide dismutase, dimethylthiourea (DMTU), diphenyl phenylene diamine (DPPD), mannitol, cyanidanol, α-tocopherol, desferoxamine, 6-hydroxy-2,5,7,8-tetramethyl chroman-2-carboxylic acid, or N-acetyl cysteine, or combinations thereof. In one embodiment, the additional antioxidant is a combination of N-acetyl cysteine, desferoxamine, and 6-hydroxy-2,5,7,8-tetramethyl chroman-2-carboxylic acid. The antioxidant agents may be present at a concentration of 0.1 mM or greater, or 1 mM or greater. The antioxidant agents may be present at a concentration of 10 mM or less, or 5.5 mM or less.

Freezing the tissue may include lowering the temperature of the tissue to a temperature below 0° C., such as to −20° C. or lower, −40° C. or lower, −50° C. or lower, −70° C. or lower, −100° C. or lower, −120° C. or lower, or −150° C. or lower. While there is no desired lower limit, in practice, tissues may be cooled to a temperature of −200° C. or greater or −180° C. or greater. In some embodiments, freezing the tissue includes vitrification. The tissue may be cooled to its target cryopreservation temperature at a set rate. For example, the tissue may be cooled at a rate of 0.05° C./min or greater, 0.1° C./min or greater, 0.5° C./min or greater, 0.8° C./min or greater, 1° C./min or greater, 5° C./min or greater, 10° C./min or greater, 20° C./min or greater, or 30° C./min or greater. The tissue may be cooled at a rate of 20° C./min or less, 15° C./min or less, 10° C./min or less, 5° C./min or less, 3° C./min or less, 2° C./min or less, 1° C./min or less, 0.5° C./min or less, or 0.1° C./min or less. In some embodiments, the tissue is cooled at a rate that is below the critical cooling rate (CCR) of the system. In some embodiments, the tissue is cooled at a rate of 0.1° C. to 5° C. per minute until the target cryopreservation temperature is reached.

The cryopreserved tissue may be rewarmed using inductive heating. Inductive heating may be induced by the inclusion of the susceptor particles in the cryopreservation solution and by subjecting the cryopreserved tissue to an alternating magnetic field. The tissue may be rewarmed at any suitable rate. In some embodiments, the tissue is rewarmed at a rate above the critical warming rate (CWR) of the system. In a preferred embodiment, the tissue is rewarmed at a rate that does not cause devitrification or crystallization of the tissue. The tissue may be rewarmed at a rate of 10° C./min or greater, 20° C./min or greater, 35° C./min or greater, 40° C./min or greater, 45° C./min or greater, or 50° C./min or greater. The tissue may be rewarmed at a rate of 100° C./min or less, 80° C./min or less, 70° C./min or less, or 60° C./min or less. In some embodiments, the tissue is rewarmed at a rate of 20° C. to 60° C. per minute until the tissue is thawed. The tissue may be rewarmed to a temperature of 0° C. or warmer, 4° C. or warmer, 10° C. or warmer, or 20° C. or warmer. The tissue may be rewarmed up to a physiological temperature (about 37° C.).

After rewarming, the tissue may be flushed to remove (e.g., unload) the cryopreservation solution. In some embodiments, the tissue is flushed using the perfusion apparatus and by perfusing the tissue with a flushing solution. The flushing solution may be free of one or more of the cryopreservative agents. The flushing may be performed stepwise by decreasing the concentration of the cryoprotective agent in the solution during the flushing process. For example, the flushing may be done in two or more steps, where the concentration of the cryoprotective agent is decreased from its concentration in the cryopreservation solution. The flushing solution may include a decreasing gradient of one or more of the cryopreservative agents. In one embodiment, flushing solution is free or substantially free of the alcohol. In one embodiment, the flushing solution includes a decreasing gradient of the sugar. For example, the concentration of the sugar may be decreased from its concentration in the cryopreservation solution to 0 M while flushing the tissue. The flushing solution may include other perfusion ingredients. For example, the flushing solution may include one or more of the components of the UW Solution with added metabolic agents, antioxidant agents, and membrane stabilizers shown in TABLE 1. The flushing may be performed at any suitable temperature about 0° C., such as 4° C. or greater, and up to about physiological temperature. According to an embodiment, the flushing removes or substantially removes the susceptor particles from the tissue. According to an embodiment, the flushing removes or substantially removes the cryopreservative agents (e.g., alcohol and sugar) from the tissue.

The method may further include digesting the tissue and recovering isolated cells from the digested tissue. For example, the tissue may be digested using collagenase. The digested tissue may further be filtered and washed. In some embodiments, the tissue is a liver or a portion of a liver, and the isolated cells are isolated hepatocytes. In some embodiments, 10⁸ cells or greater, 10⁹ cells or greater, 10¹⁰ cells or greater, 10¹¹ cells or greater, or 10¹² cells or greater may be isolated from a single tissue specimen.

In some embodiments, the method may include slicing the tissue after rewarming. Slicing may be performed using any known method, such as by using a slicer. In one embodiment, the method includes slicing a rewarmed liver.

In some embodiments, the method may further include removing an organ from a donor that has suffered cardiac arrest; circulating the cryopreservation solution including a perfusion solution, a cryopreservative agent, and susceptor particles through the organ under hypothermic conditions (e.g., between 0° C. and 7° C.); freezing the organ; and inductively rewarming the organ.

The method may include, prior to circulating the cryopreservation solution, flushing the organ with a solution to remove any blood or residual material from within the organ. The flush solution may have a concentration of K′ ions similar to that of plasma (e.g., about 4.5 mM). A suitable flush solution may be a Krebs-Henseleit buffer solution or similar plasma-like salt solution. After flushing is complete, the organ may be placed on the perfusion apparatus and cooled over the course of 3 to 5 minutes by perfusion with cold flush solution. Once the organ is at hypothermic temperature, the organ can be perfused with the cryopreservation solution.

In some embodiments the perfusion of the tissue with the cryopreservation solution also includes continuous administration of oxygen. The partial pressure of oxygen in the cryopreservation solution may be 100 mmHg or greater or 150 mmHg or greater. The partial pressure of oxygen in the cryopreservation solution may be 175 mmHg or less.

In some embodiments, a method of preserving tissue (for example, a portion of an organ or a whole organ) includes loading the tissue with a solution comprising a cryoprotective agent and susceptor particles; freezing the tissue; thawing the tissue; and after freezing and thawing, further processing the tissue. Further processing may include digesting the tissue and recovering isolated cells from the digested tissue. Further processing may include slicing the tissue. The tissue may include at least a portion of an organ. The tissue may include a whole organ. The tissue may include at least a portion of a liver. The tissue may include a whole liver. The loading of the solution may be done via the vasculature of the tissue. The tissue may be thawed using inductive heating. The susceptor particles may include nanoparticles.

In some embodiments, a method of preserving tissue (for example, a portion of an organ or a whole organ) includes loading the tissue with a solution comprising a cryoprotective agent and susceptor particles; freezing the tissue; thawing the tissue; and after freezing and thawing, further processing the tissue. The loading of the solution may be done via the vasculature of the tissue. The loading of the solution may be done stepwise or using a gradient where the concentration of cryoprotective agents is increased. The temperature of the tissue or the solution or both the tissue and the solution may be lowered as the concentration of the cryoprotective agent is increased. Susceptor particles may be added at the end of the loading, such as during a final loading step.

In some embodiments, a method of preserving tissue may include perfusing a biospecimen comprising at least a portion of an organ; loading the biospecimen with a solution comprising a cryoprotective agent and susceptor particles; freezing the biospecimen; thawing the biospecimen by inductively heating the susceptor particles; and after freezing and thawing, further processing the biospecimen. Further processing may include digesting the biospecimen and recovering isolated cells from the digested biospecimen. Further processing may include slicing the biospecimen. The biospecimen may be a whole organ. The organ may be a liver. The loading of the solution may be done via the vasculature of the organ. The perfusing may be done using a perfusion solution. The perfusion solution may include one or more of a membrane stabilizer, a metabolic support agent, and an antioxidant. The perfusion solution may include a cryoprotective agent. The perfusion solution may also include susceptor particles and may be used to load the susceptor particles into the biospecimen. The susceptor particles may include nanoparticles.

In some embodiments, a method of preserving tissue (for example, a portion of an organ or a whole organ) may include loading the tissue with a solution comprising a cryoprotective agent and susceptor particles prior to freezing and inductively thawing the tissue. The cryoprotective agent may include one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof. The cryoprotective agent may be present at a concentration of 1M to 10 M. The alcohol may be present at a concentration of 0.5 M to 10 M, or 1 M to 8 M. The sugar may be present at a concentration of 0 M to 3 M, or 0.1 M to 2 M. The cryoprotective agent may include 1 M to 8 M alcohol and 0 M to 2 M sugar. The cryoprotective agent may include 1 M to 8 M ethylene glycol and 0.1 M to 2 M sucrose.

In some embodiments, a method of preserving tissue may include perfusing a biospecimen comprising at least a portion of an organ with a perfusion solution; loading the biospecimen with a solution comprising a cryoprotective agent and susceptor particles; freezing the biospecimen; and thawing the biospecimen by inductively heating the susceptor particles. The perfusion solution may include from 0.1 mM to 10 mM of a membrane stabilizer; from 0.1 mM to 15 mM of a metabolic support agent; from 0.1 mM to 10 mM of an antioxidant agent selected from the group consisting of beta-carotene, catalase, superoxide dismutase, dimethyl thiourea (DMTU), diphenyl phenylene diamine (DPPD), mannitol, cyanidanol, a-tocopherol, desferoxamine, 6-hydroxy-2,5,7,8-tetramethyl chroman-2-carboxylic acid, and N-acetyl cysteine, and combinations thereof; a cryoprotective agent; and susceptor particles. The susceptor particles may include nanoparticles. The susceptor particles may include iron oxide. The susceptor particles may include iron oxide nanoparticles. The cryoprotective agent may be present at a concentration of 1M to 10 M and include one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof. The biospecimen may be loaded with a cryopreservation solution (e.g., vitrification solution) including one or more of an alcohol and a sugar. The alcohol may be selected from glycerol, sorbitol, ethylene glycol, propylene glycol, inositol, xylitol, mannitol, arabitol, ribitol, erythritol, threitol, galactitol, pinitol, and combinations thereof. The alcohol may be present at a concentration of 0.5 M to 10 M or 1 M to 8 M. The sugar may be selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof. The sugar may be present at a concentration of 0 M to 2 M or from 0.1 M to 1 M. The alcohol may be glycerol, ethylene glycol, or propylene glycol. In some embodiments the alcohol is ethylene glycol and the sugar is sucrose. In one embodiment, the cryopreservation solution includes 1 M to 8 M ethylene glycol and 0.1 M to 2 M sucrose.

### EXAMPLES

The use of cryoprotective agents and susceptor particles in cryopreservation and subsequent inductive rewarming was tested on whole livers.

### Example 1

Several experiments were conducted on whole rat livers. First, a vitrification solution was loaded and unloaded in the liver and hepatocytes were isolated and assessed for yield, viability and functionality. Second, the livers were loaded and unloaded with the vitrification solution and susceptor particles to assess the distribution, location of the susceptor particles in the liver and washout. Next, vitrification solution and susceptor particles loaded livers were cooled to −150° C. to assess vitrification and cooling rates. The livers were then inductively rewarmed using inductive heating to determine warming rates and temperature variation in the liver. Finally, the vitrification solution was unloaded and the inductively rewarmed liver was normothermically re-perfused for functional and flow assessment. All loading and unloading steps were done at 4° C. The yield, viability, and functionality of hepatocytes isolated from vitrification solution loaded and unloaded livers was compared with fresh controls.

The vitrification solution included 40 wt-% Ethylene Glycol (EG) and 0.6 M sucrose. The total concentration of the cryoprotective agents was 7 M. The susceptor particles were silica-coated iron oxide nanoparticles with an approximate hydrodynamic diameter of 80 nm. The concentration of the susceptor particles, expressed in mass of iron per milliliter, was 10 mg Fe/mL. The nanoparticles are further described in Gao, Z. et al. (2020) Preparation of Scalable Silica-Coated Iron Oxide Nanoparticles for Nanowarming. Advanced Science, 1901624.

The loading was done on a customized temperature-controlled perfusion system for isolated livers, available from Radnoti, LLC in Covina Calif. The vitrification solution was loaded in 3 steps with 10 wt-% EG, 25 wt-% EG, and 40 wt-% EG+0.6 M sucrose, respectively. Each step had a duration of 5 min (group 1, n=1) or 15 min (group 2, n=2). The vitrification solution was unloaded in 5 steps using 1 M sucrose, 0.7 M sucrose, 0.5 M sucrose, 0.25 M sucrose, and 0 M sucrose with 5 min and 10 min durations, respectively. The carrier solution was a HepatoSys solution developed as a resuscitating and hypothermic preservation solution, available from HepatoSys, Inc. in Cornelius, N.C.

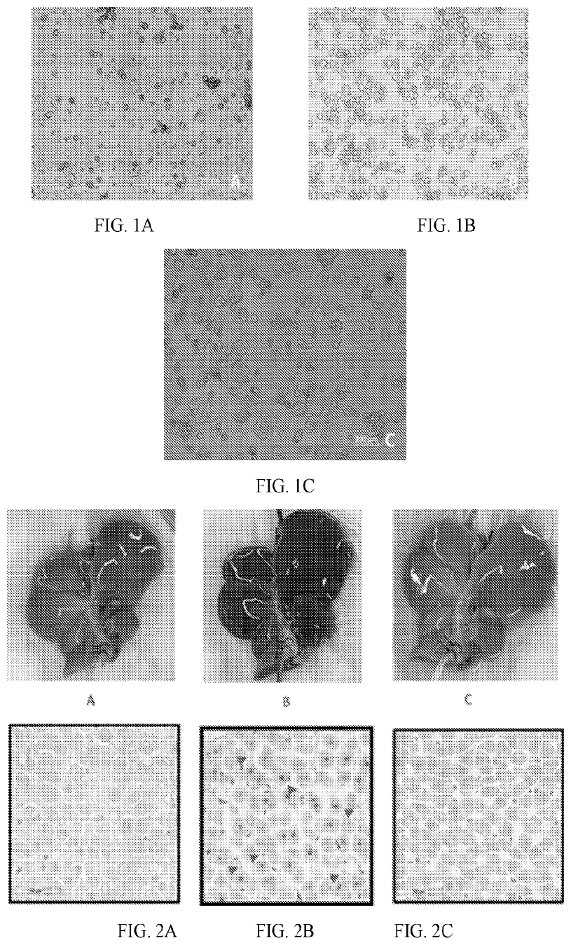

Hepatocytes were isolated and plated at a density of 3×10⁶ cells per well in 6-well plates. Viability for controls, group 1, and group 2 were 92.8%±1.6%, 87.25%, and 87.07%±0.73%, (mean±standard error) respectively. Yield for group 2 was 95.5% of the controls, while group 1 yield was higher than the controls, possibly due to only having a n=1. FIGS. 1A-1C shows images of plated hepatocytes from the control and experimental groups. All groups displayed classic hepatocyte morphology spreading of the cytoplasm and some multi-nuclei cells.

Cells of samples from groups 1 and 2 and the control were subjected to a cytochrome P450 study. Cytochrome P450 (CYP) reactions are commonly used to assess drug metabolism. The study was performed using a 7-ethoxycoumarin 0-deethylation (ECOD) assay (n=3 wells). After 1-day induction with 10 nM 2,3,7,8-tetrachlorodibenzo-p-dioxin (TCDD), 7-ethoxycoumarin (100 μM) was incubated with cells for 60 mins and the fluorescence was measured. 7-hydroxycoumarin glucuronide and 7-hydroxycoumarin sulfate were hydrolyzed by a glucuronidase/arylsulfatase acetate solution to 7-hydroxycoumarin for 90 mins and reassessed. For control cells, Phase I and II activities were determined to be 16.1±3.5 and 8.8±2.5 pmol/min/10⁶ cells, respectively. Although group 1 and 2 had Phase I values of 13.5 and 10.6 pmol/min/10⁶ cells, respectively, Phase II values were lower at 5.1 and 3.0 pmol/min/10⁶ cells. The Phase I and II results indicate that Cytochrome P450 activities, important in drug metabolism, are maintained after the loading and unloading of the cryoprotectants.

The results suggest that loading and unloading of the vitrification solution leads to high yield and viability of functioning hepatocytes in a rat liver. In addition, the results suggest that the whole liver can be vitrified after vitrification solution and susceptor particle loading. The ability to provide a broad range of high quality primary human cells will have an immediate impact in such fields as cell transplantation, bioartificial organs, and engineered organs. In addition, this technology can provide donor pool cells to better reflect the general population response to treatments.

### Example 2

Whole rat livers were loaded and unloaded with the vitrification solution and susceptor particles to assess the distribution, location of the susceptor particles and susceptor particles washout (n=2) using group 2 protocol described above in Example 1 (with 15 min loading steps and 10 min unloading steps). The susceptor particles were loaded as part of the last loading step (during final 2 mins).

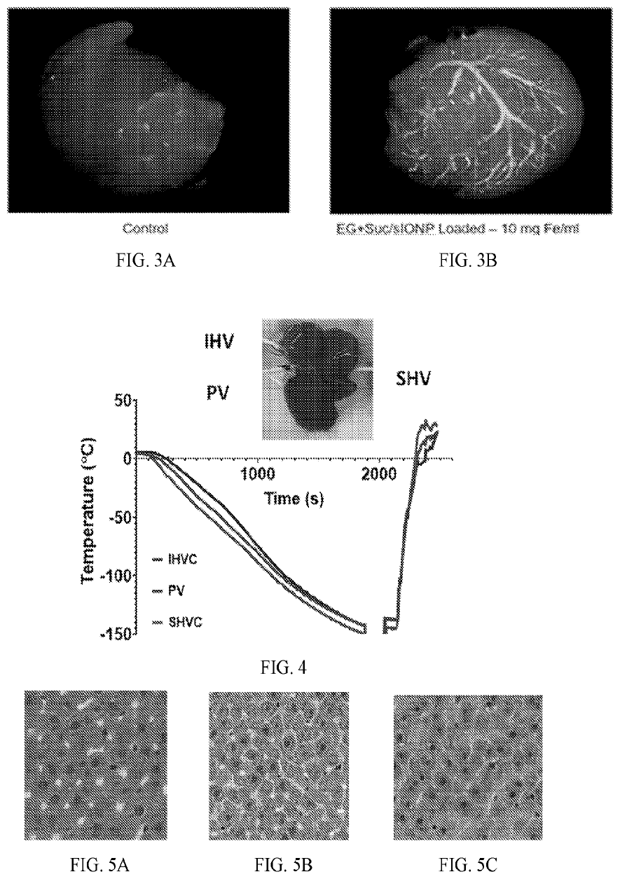

Histology images of a liver before loading of the vitrification solution, with vitrification solution and susceptor particles loaded, and with vitrification solution and susceptor particles unloaded are shown in FIGS. 2A, 2B, and 2C, respectively. The histology image shown in FIG. 2B shows that the distribution of the susceptor particles was uniform throughout the liver, which also suggest the same for the distribution of the vitrification solution. FIG. 2B shows that the susceptor particles (blue) reside in the extracellular space and are not taken up by the hepatocytes and other nonparenchymal cells. This is also confirmed by micro-CT images of susceptor particles in the vasculature, as shown in FIG. 3B. Although the washout image (FIG. 2C) shows complete removal of the susceptor particles, SWIFT MRI shows that small residual amount of susceptor particles do remain in the livers (data not shown).

### Example 3

Whole rat livers were loaded with a vitrification solution and susceptor particles, vitrified, and inductively rewarmed, to assess whether recrystallization could be avoided.

The livers were loaded with vitrification solution and susceptor particles as described above in Example 2. Livers (n=2) were placed in plastic bags filled with the vitrification solution and susceptor particle solution. Fiber optic temperature probes were placed into the portal vein (PV), intrahepatic vena cava (IHVC), and suprahepatic vena cava (SHVC) cannulas. The IHVC probe was inserted 1 cm beyond the end of the cannula to monitor the temperature closer to the interior of the liver. The livers were placed in a control-rate freezer at −150° C.

The livers achieved cooling rates between 5.3-6.1° C./min (shown in FIGURE. 4), suggesting that they were sufficient to achieve vitrification of the liver. No visible cracking due to thermal stress was seen.

The livers were inductively rewarmed in a 15 kW RF alternating magnetic field system and warming rates averaged 57° C./min (shown in FIG. 4). Variation in temperatures measured at the three locations were ˜5° C. No recrystallization or cracking was observed during periodic observation of the liver during inductively rewarming.

Histology images of the control liver, vitrification-solution-loaded and unloaded liver, and vitrified and inductively rewarmed liver are shown in FIGS. 5A, 5B, and 5C, respectively.

### Example 4

A whole rat liver was loaded with a vitrification solution and susceptor particles, vitrified, and inductively rewarmed, to assess whether recrystallization could be avoided.

The liver was loaded with vitrification solution and susceptor particles as described above in Example 2. The liver was vitrified to −150° C., inductively rewarmed at a rate of approximately 55-60° C./min, and normothermically reperfused for 30 mins. Indocyanine green (ICG, 25 μg/ml, specifically taken up by hepatocytes) was premixed into the perfusate. The perfusate was a Krebs-Henseleit buffer.

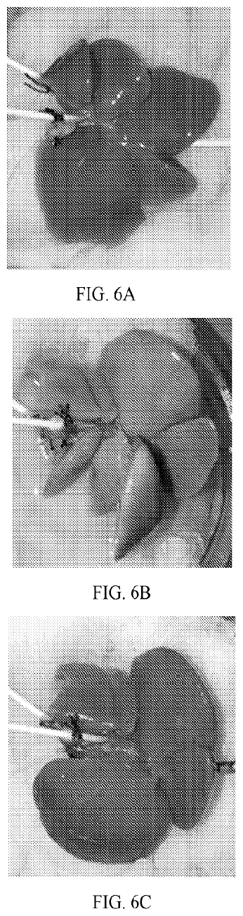

Perfusate effluent was collected every 15 min to determine ICG quantity (780 nm). The effluent samples showed 52.5% and 55.6% clearance from the buffer at 15 and 30 mins, respectively. Images of a liver during normothermic re-perfusion with ICG at 0 minutes, 15 minutes, and 30 minutes after vitrification, inductive rewarming, and unloading of the vitrification solution are shown in FIGS. 6A, 6B, and 6C, respectively. The liver in FIG. 6A exhibits a normal brown liver color. The liver in FIG. 6B exhibits a light to medium green color. The liver in FIG. 6C exhibits a strong medium green color. Based on the color of the liver seen in the images, ICG is well distributed throughout the liver indicating a homogeneous flow during reperfusion. The disappearance of the ICG from the perfusate and appearance in the liver would indicate hepatocyte specific function with the concentrated ICG most likely appearing in the bile canaliculi. Both conditions, homogeneous flow and hepatocyte specific function, indicate that hepatocytes can be isolated from livers that have been vitrified and inductively rewarmed.

Taken together, these results suggest that there is potential for this project to produce large quantities of viable and functional hepatocytes isolated from vitrified and inductively rewarmed livers.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A method of preserving tissue, the method comprising:
loading the tissue with a solution comprising a cryoprotective agent and susceptor particles;
cryopreserving the tissue;
rewarming the tissue; and
processing the tissue
after the cryopreserving and the rewarming.

2. The method of claim 1, wherein the tissue comprises at least a portion of a liver.

3. The method of claim 1, wherein the tissue comprises a whole organ.

4. The method of claim 1, wherein the tissue comprises at least portion of an organ and the loading of the solution is done via the vasculature of the organ.

5. The method of claim 1, wherein the cryoprotective agent comprises one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof.

6. The method of claim 1, wherein the cryoprotective agent is present at a concentration of 1M to 10 M.

7. The method of claim 1, wherein the tissue is rewarmed using magnetically induced heating of the susceptor particles.

8. The method of claim 1, wherein the susceptor particles comprise nanoparticles.

9. (canceled)

10. The method of claim 19, wherein the processing the tissue comprises digesting the tissue and recovering isolated cells from the digested tissue.

11. The method of claim 1, wherein the processing the tissue comprises slicing the tissue and/or isolating sections of the tissue.

12-16. (canceled)

17. A method of preserving tissue, the method comprising:
perfusing a biospecimen comprising at least a portion of an organ;
loading the biospecimen with a solution comprising a cryoprotective agent and susceptor particles;
cryopreserving the biospecimen;
rewarming the biospecimen by magnetically induced heating of the susceptor particles; and
processing the biospecimen after the cryopreserving and the rewarming.

18. The method of claim 17, wherein the organ comprises a liver.

19. The method of claim 17, wherein the biospecimen comprises a whole organ.

20. The method of claim 17, wherein the loading of the solution is done via the vasculature of the organ.

21. The method of claim 17, wherein the cryoprotective agent comprises one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof.

22. The method of claim 17, wherein the cryoprotective agent is present at a concentration of 1M to 10 M.

23. The method of claim 17, wherein the susceptor particles comprise nanoparticles.

24. (canceled)

25. The method of claim 17, wherein the processing the biospecimen comprises digesting the rewarmed biospecimen and recovering isolated cells from the digested biospecimen.

26. The method of claim 17, wherein the processing the biospecimen comprises slicing the biospecimen and/or isolating sections of the biospecimen.

27-30. (canceled)

31. A perfusion solution comprising:
from 0.1 mM to 10 mM of a membrane stabilizer;
from 0.1 mM to 15 mM of a metabolic support agent;
from 0.1 mM to 10 mM of an antioxidant agent selected from the group consisting of beta-carotene, catalase, superoxide dismutase, dimethyl thiourea (DMTU), diphenyl phenylene diamine (DPPD), mannitol, cyanidanol, a-tocopherol, desferoxamine, 6-hydroxy-2,5,7,8-tetramethyl chroman-2-carboxylic acid, and N-acetyl cysteine, and combinations thereof;
a cryoprotective agent; and
susceptor particles.

32. The perfusion solution of claim 31, wherein the susceptor particles comprise nanoparticles.

33. (canceled)

34. The perfusion solution of claim 31, wherein the cryoprotective agent is present at a concentration of 1M to 10 M and comprises one or more of an alcohol and a sugar, wherein the alcohol is selected from glycerol, sorbitol, ethylene glycol, propylene glycol, inositol, xylitol, mannitol, arabitol, ribitol, erythritol, threitol, galactitol, pinitol, and combinations thereof; and the sugar is selected from sucrose, trehalose, maltose, lactose, fructose, glucose, dextran, melezitose, raffinose, nigerotriose, maltotriose, maltotriulose, kestose, cellobiose, chitobiose, lactulose, and combinations thereof.
